(19) United States
(12) Patent Application Publication
(10) Pub. No.: US 2009/0311227 A1
(43) Pub. Date: Dec. 17, 2009

(54) Composition

(75) Inventors: Arthur Ouwehand, Inkoo (FI); Pierre Desreumaux, Monceau (FR); Didier Carcano, Paris (FR)

Correspondence Address: STEPTOE & JOHNSON LLP, 1330 CONNECTICUT AVENUE, N.W., WASHINGTON, DC, 20036, US

(73) Assignee: Danisco A/S, Copenhagen K (DK)

(21) Appl. No.: 12/300,283

(22) PCT Filed: May 11, 2007
(86) PCT No.: PCT/IB2007/002606

§ 371 Date: May 12, 2009

Related U.S. Application Data
(60) Provisional application No. 60/800,138, filed on May 12, 2006.

(51) Int. Cl. A61K 35/74; A61P 29/00

(52) U.S. Cl. 424/93.45

(57) ABSTRACT

The present invention relates to a composition comprising: i) at least one strain of a Lactobacillus sp and/or a metabolite thereof; and ii) a cannabinoid receptor agonist and/or a opioid receptor agonist. In addition, the present invention relates to methods using and uses of such a composition.

### FIELD OF INVENTION

The present invention relates to compositions (such as analgesic compositions), methods of using such compositions and uses thereof.

In particular, the present invention relates to a composition comprising at least one strain of Lactobacillus in combination with an opioid receptor agonist and/or a cannabinoid receptor agonist as well as uses thereof.

### BACKGROUND TO THE INVENTION

In order to meet the requirements of growing and ageing populations, it has become necessary to provide an efficient solution to decrease or eliminate pain which can be expressed as discomfort or even as intense suffering. Pain can be caused by inflammation but can also exacerbate or increase inflammation.

For example, abdominal pain is a common symptom attributed to visceral hypersensitivity.

To date, some opioid receptor agonists (such as morphine) and cannabinoid receptor agonists (such as cannabis) have been used for pain relief.

The opioid receptors δ, κ and μ belong to the superfamily of receptors coupled to the G proteins which are composed of seven transmembrane helices. The intracellular part of the receptor is in contact with the G protein which is associated with it and which can vary depending on the type of agonists used. The opioid receptors, in particular the μ receptor, have several functions. The main one is an analgesic role demonstrated by the use of β-endorphin- or morphine-type agonists specific to this receptor passing the haematomeningeal barrier. The second function of this receptor in the digestive tract is to reduce the intestinal transit by inhibiting secretion and digestive motricity. Thirdly, the opioid receptors are also involved in the regulation of intestinal inflammation. The μ receptor for the opioids is present in the central nervous system but also at the periphery. Its presence has been detected in the majority of the vital organs of the human body: the spleen, liver, kidneys, small intestine and colon in particular in the intestinal nervous system in the neurons of the submucous and mesenteric plexus, but also, in vitro, in the lymphocytes, monocytes/macrophages and epithelial cells.

The cannabinoid receptors, called CB 1 and CB2, belong to the superfamily of receptors coupled to the G proteins which are composed of seven transmembrane helices. They are expressed essentially by the central and peripheral nervous system for CB1 and the immune response cells for CB2. In humans, there are two endogenous ligands of these cannabinoid receptors, which are naturally produced by the intestinal epithelial cells.

The cannabinoid receptors CB1 expressed by the enteric nervous system would be the cause of a slowing-down of the peristalsis of the stomach and small intestine and an inhibition of gastric secretion. Other anti-diarrheic and anticancer functions of the cannabinoid receptors are presumed.

For the above reasons opioid receptor agonists and cannbinoid receptor agonists have been used. However, such agonists have side effects. For instance, cannbinoid receptor agonists have psychological side effects (such as altered memory, amnesia, hallucination, psychotropic effects, sedation, euphoria, anxious depression, fear of death and fear of losing control etc.) and physical side effects (such as movement disorders, muscular weakness, speech disorders and alertness disorders etc.). Likewise, opioid receptor agonists have side effects including: nausea, retention of faeces, vomiting, respiratory depression, withdrawal syndrome and alertness disorders.

In addition, the use of such opioid and cannabinoid receptor agonists may be expensive and can lead to dependency on the agonists.

Accordingly, it is an object to provide compositions which can be used to relieve, prevent and/or treat conditions usually treated by opioid and/or cannbinoid receptor agonists (such as pain, intestinal inflammation, diarrhea and/or cancer) whilst reducing the side effects, costs and/or risk of dependency resulting from the use of opioid and/or cannbinoid receptor agonists.

### SUMMARY OF THE INVENTION

The present invention has surprisingly found that at least one strain of Lactobacillus and/or a metabolite thereof enhances the activity of cannabinoid receptor agonists and/or a opioid receptor agonists. Accordingly, the present invention provides a composition comprising at least one strain of Lactobacillus and/or metabolite thereof in combination with a cannabinoid receptor agonist and/or an opioid receptor agonist.

### SUMMARY ASPECTS OF THE PRESENT INVENTION

In one aspect, the present invention relates to a composition (such as an analgesic composition and/or pharmaceutical composition) comprising: i) at least one strain of a Lactobacillus sp and/or a metabolite thereof; and ii) a cannabinoid receptor agonist and/or an opioid receptor antagonist.

In another aspect, the present invention relates to a method of treating and/or ameliorating any one or more of the group selected from: pain, inflammation (such as intestinal inflammation and/or inflammatory bowel disease), neoplasism and diarrhea comprising administering an effective amount of a composition comprising: i) at least one strain of a Lactobacillus sp and/or a metabolite thereof; and ii) a cannabinoid receptor agonist and/or a opioid receptor antagonist.

In a further aspect of the present invention, there is provided the use of a composition comprising: i) at least one strain of Lactobacillus sp and/or a metabolite thereof; and ii) a cannabinoid receptor agonist and/or a opioid receptor antagonist as one or more of the following selected from the group consisting of: an analgesic, an anti-inflammatory, an anti-diarrheic and an anti-neoplastic.

In another aspect, the present invention relates to the use of a composition comprising: i) at least one strain of Lactobacillus sp and/or a metabolite thereof; and ii) a cannabinoid receptor agonist and/or an opioid receptor antagonist in the manufacture of a medicament for treating and/or ameliorating the perception of pain.

In a further aspect, the present invention relates to the use of a composition comprising: i) at least one strain of Lactobacillus sp and/or a metabolite thereof; and ii) a cannabinoid receptor agonist and/or an opioid receptor antagonist in the manufacture of a medicament for treating and/or ameliorating diarrhea.

In another aspect, the present invention relates to the use of a composition comprising: i) at least one strain of Lactobacillus and/or a metabolite thereof; and ii) a cannabinoid receptor agonist and/or an opioid receptor antagonist in the manufacture of a medicament for treating and/or ameliorating inflammation (such as intestinal inflammation and/or inflammatory bowel disease).

In a further aspect, the present invention relates to the use of a composition comprising: i) at least one strain of Lactobacillus sp and/or a metabolite thereof; and ii) a cannabinoid receptor agonist and/or a opioid receptor antagonist in the manufacture of a medicament for treating and/or ameliorating irritable bowel syndrome and/or irritable bowel disease.

In yet another aspect, the present invention relates to the use of at least one strain of a micro-organism (such as Lactobacillus) and/or at least one metabolite thereof to prepare a support administered to humans or animals suitable for reducing undesirable side effects of cannabinoid and/or opioid receptor agonists.

The term “analgesic” as used herein refers to a relief of pain. Suitably the relief of pain may be a neurologic or pharmacologic state in which painful stimuli are so moderated that, though still perceived, they are no longer painful. Thus, an “analgesic composition” is able to reduce the perception of pain.

The term “nociceptive” as used herein refers to being capable of the appreciation or transmission of pain.

The term “antineoplastic” as used herein refers to the prevention and/or reduction of the development, maturation or spread of neoplastic cells. An “antineoplastic composition” may be used for the prevention, treatment and/or amelioration of cancer growth.

### ADVANTAGES

Suitably the combined use of morphine (or of other cannabinoid or opioid receptor agonists) and at least one strain of Lactobacillus and/or a metabolite thereof according to the invention may enhance the effect of morphine (or of other cannabinoid or opioid receptor agonist). This synergetic effect may advantageously lead to the use of lower dosage of morphine or of other cannabinoid or opioid receptor agonists.

Another advantage is that this treatment is may result in reduced side effects of the cannabinoid and/or opioid receptor agonists.

The compositions of the present invention may also be cheaper than the use of cannabinoid and/or opioid receptor agonists alone due to the lower dose of cannabinoid and/or opioid receptor agonists required.

### DESCRIPTION OF THE PREFERRED EMBODIMENTS

We demonstrate that oral administration of at least one strain of Lactobacillus sp and/or a metabolite thereof, for example specific Lactobacillus strains and their metabolites, induces expression of pain receptors in the gut and more specifically μ opioid and cannabinoid receptors by intestinal epithelial cells, and mediates analgesic functions in the gut which are equivalent to pharmaceutical levels morphine effects. These results suggest that microbiology of organ tissues, for example the intestinal tract, influences our internal body perception and opens new perspectives in the management of pain, for example abdominal pain, irritable bowel syndrome (IBS) and irritable bowel disease (IBD).

To this end of providing an efficient solution to decrease or eliminate pain, the present invention proposes the use at least one strain of Lactobacillus sp and/or a metabolite thereof in combination with cannabinoid and/or opioid receptor agonists.

### At Least One Strain of Lactobacillus

Amongst the strains of Lactobacilli which can be used, probiotic strains are preferred.

The term “probiotic” as used herein defines a viable strain which is capable of beneficially affecting the host organism by improving its intestinal microbial balance. The term “probiotic” as used herein also encompasses a viable strain that can stimulate the beneficial branches of the immune system and at the same time decrease most of the inflammatory reactions in the gut. In this regard, the use of the composition of the present invention, containing said probiotic ingredient for anti-cancer therapy and prevention of allergies and ulcerative colitis is also contemplated.

Suitably strains that may be used in the present invention include probiotic strains of Lactobacillus acidophilus (such as L. acidophilus NCFM), Lactobacillus salivarius (such as L. salivarius LS 33), and Lactobacillus casei.

At least one strain of Lactobacillus acidophilus may be used. Suitably, the at least one strain of Lactobacillus acidophilus may be Lactobacillus acidophilus PTA-4797. This strain of Lactobacillus acidophilus has been deposited by Rhodia Chimie, 26, quai Alphonse Le Gallo, 92 512 BOULOGNE-BILLANCOURT Cedex France, in accordance with the Budapest Treaty on 15 Nov. 2002 at the American Type Culture Collection (ATCC) 10801 University Blvd. Manassas, Va. 20110-2209, United States of America, where it is recorded under registration number PTA-4797. This strain is known as Lactobacillus acidophilus NCFM (referred to as “NCFM” below).

In addition and/or in the alternative at least one strain of Lactobacillus salivarius may be used. Suitably, the at least one strain of Lactobacillus salivarius may be Lactobacillus salivarius PTA-4800. This strain of Lactobacillus salivarius has been deposited by Rhodia Chimie, 26, quai Alphonse Le Gallo, 92 512 BOULOGNE-BILLANCOURT Cedex France, in accordance with the Budapest Treaty on 15 Nov. 2002 at the American Type Culture Collection (ATCC) 10801 University Blvd. Manassas, Va. 20110-2209, United States of America, where it is recorded under registration number PTA-4800. This strain is known as Lactobacillus salivarius LS 33 (referred to as “LS 33” below).

The at least one strain of Lactobacillus may be a naturally occurring Lactobacillus or it may be a transformed Lactobacillus. The at least one strain of Lactobacillus may also be a combination of suitable Lactobacillus strains.

It is to be understood that where reference is made in the present specification, including the accompanying claims to ‘a’ at least one strain of Lactobacillus strain or ‘a’ metabolite thereof, such reference is meant to include one or more strain of Lactobacillus or one or more metabolites thereof, and mixtures thereof, unless it is specifically stated otherwise in the text.

Preferably the at least one strain of Lactobacillus is capable of normal growth and development. In some embodiments, the at least one strain of Lactobacillus may be killed.

As used herein the term “transformed” encompasses recombinant Lactobacilli. The term “recombinant Lactobacillus” means a Lactobacillus which carries a recombinant nucleotide sequence coding for an exogenous gene. The transformed Lactobacillus may also have the capacity for example to utilise different enzyme substrates as a carbon source, to ferment at a different temperature range, exhibit resistance to bacteriophage attack, be capable of quicker capacity to replicate following rehydration in the gut when compared to the parent. Advantageously a recombinant Lactobacillus may be able to have an increased tolerance to the low pH values experienced in the gut of the consumer on its route to the lower gut.

### Metabolites

Without wishing to be bound by theory, it is believed that the soluble metabolites associated with, for example produced by, the Lactobacillus strain may be causing the advantageous effects of the Lactobacillus strain. For some aspects, it may therefore be unnecessary for the Lactobacillus strain to be in direct contact with the target cells. For some aspects, it is believed that one or more metabolites associated with, for example produced by, the Lactobacillus strain may be suitable for achieving the beneficial effects taught herein. In such instances, it may be unnecessary to include the Lactobacillus strain in the compositions of the present invention. The term “metabolite thereof” as used herein means one or more compounds either extracted from the Lactobacillus strain according to the present invention or obtained from a culture medium in which a Lactobacillus strain according to the present invention is or was cultured. In some aspects the metabolite may be a crude extract of the culture medium and/or Lactobacillus strain. Suitably, for some aspects the metabolite may be one or more compounds isolated and/or purified from the culture medium and/or from the Lactobacillus strain (such as a compound produced by the microorganisms or DNA, RNA or pieces of cell envelope, i.e. cell membrane and cell wall, from microorganisms). In one embodiment, metabolites may also be killed microorganisms.

### Conditions

The use of at least one strain of Lactobacillus and/or at least one metabolite thereof to prepare a support administered to humans or animals suitable for inducing an effect similar to cannabinoid or opioid receptor agonists is made in combination with cannabinoid or opioid receptor agonists.

Suitable a composition of the present invention may be used as any one or more selected from the group consisting of: an analgesic, unaesthetic, anti-inflammatory, antidiarrheic, a metabolic regulator and an antineoplastic.

Suitably compositions comprising at least on strain of Lactobacillus and/or a metabolite thereof in combination with an opioid receptor agonist and/or cannbinioid receptor agonists may be synergistic compositions.

The synergic effect is particularly efficient in the case of decrease or elimination of pain in the case of cancer, specifically colon cancers.

Suitably, an analgesic or an anaesthetic or an anti-inflammatory or an antidiarrheic effect or a regulatory effect on the metabolism or an antineoplasic effect may be mediated via the opioid receptors or cannabinoid receptors.

Suitably, the analgesic effect is particularly efficient in case of irritable bowel syndrome (IBS) and irritable bowel disease (IBD).

The analgesic or the anaesthetic or the antidiarrheic effect is preferably provided via opioid receptors.

The anti-inflammatory effect is preferably provided via both opioid receptor MOR and cannabinoid receptors CB1 and CB2.

The regulatory effect on the metabolism is preferably provided via cannabinoid receptors CB1 and CB2.

The antineoplasic effect metabolism is preferably provided via cannabinoid receptor CB1 and MOR.

### Support

The compositions of the present invention may comprise a support.

Suitably the support may be a pharmaceutically acceptable support or a food product.

In one embodiment, the support may be administrated orally.

Oral forms can also comprise syrup, tablet, capsule, lotion, patch, confectionary, candy, lozenge and other classic pharmaceutical oral forms.

In another embodiment, the support can be administrated subcutaneously.

In another embodiment, the mode of administration of the support is mucosal, for example cutaneous, oral (mouth mucosa), pulmonary, nasal, rectal, ocular, urogenital or vaginal.

The support may be administered under the form of pomade or ointment (for cutaneous application for example), aerosol (e.g. nasal or pulmonary applications), enema (e.g. rectal or vaginal application), lotion or patch (e.g. mouth mucosa application) or other classic pharmaceutical forms.

Nevertheless, the invention encompasses as well the systemic effect of the composition of the present invention which, being for example injected subcutaneously can have an effect on a remote place, for example in the intestines.

In another embodiment, the support administered is a dairy product of animal or vegetable origin.

### Administration

Typically, a physician will determine the actual dosage of the composition which will be most suitable for an individual subject and it will vary with the age, weight and response of the particular subject. The dosages below are exemplary of the average case. There can, of course, be individual instances where higher or lower dosage ranges are merited.

Preferably, the actual dosage that is used results in minimal toxicity to the subject.

In one embodiment of the invention, the composition has an effect similar to 0.1 to 4 mg of morphine per kilogram of human or animal body, preferably 0.5 to 2 mg of morphine per kilogram of human or animal body, and more preferably around 1 mg of morphine per kilogram of human or animal body.

The compositions of the present invention may be administered by direct injection. The composition may be formulated for parenteral, mucosal, intramuscular, intravenous, subcutaneous, intraocular, intradermal or transdermal administration.

Suitably, the at least one strain of Lactobacillus may be present in the compositions according to the present invention at a dose of 10⁶ to 10¹⁴ CFU, preferably from 10⁸ to 10¹² CFU and may be administered as a dingle dose or repeated doses may be administered at determined intervals.

The invention also proposes a method for inducing an effect similar to the effect of cannabinoid or opioid receptor agonists comprising repeatedly administering to a human or an animal an effective amount of Lactobacillus strain(s). Preferably, from 10⁶ to 10¹⁴ CFU, preferably from 10⁸ to 10¹² CFU per day of lactic acid bacteria strains is administered.

Suitably, the composition may be administered for at least one day, preferably at least two days, preferably at least 5 days, preferably for about 15 days.

Preferably between from 10⁹ to 10¹¹ CFU per day of 1 Lactobacillus strain(s) is administered.

The administration to a human or an animal of an effective amount of lactic acid bacteria strains is performed in combination with the administration of cannabinoid and/or opioid receptor agonists.

Opioid receptor agonists can be morphine but also morphine derivatives and every natural or synthetic ligand which bind to the μ opioid receptor MOR and which is classically used as a pain-killer. Such compounds are known to a person of ordinary skill in the art. They can also be antidiarrheics such as Immodium®. Cannabinoid receptor agonists can be cannabis derivatives and every natural or synthetic ligand which bind to cannabinoid receptors CB1 and/or CB2 and which is classically used as a pain-killer. Such compounds are known to a person of ordinary skill in the art.

The microorganism and/or metabolite thereof may be administered as a dairy product of animal or vegetable origin. The methods of use of the invention involve administration of at least one microorganism and/or at least one metabolite thereof to humans or animals in amounts suitable for inducing an effect similar to cannabinoid or opioid receptor agonists in combination with cannabinoid or opioid receptor agonists.

The methods of the invention may involve administration of to humans or animals comprising from 10⁶ to 10¹⁴ CFU, preferably 10⁸ to 10¹² CFU of the microorganisms.

The invention is also directed to methods for inducing an effect similar to the effect of cannabinoid or opioid receptor agonists comprising repeatedly administering to a human or a animal an effective amount of microorganisms and/or at least one metabolite thereof preferably lactic acid bacteria strains and/or probiotic strains and more preferably Lactobacillus strains. Preferably, from 10⁶ to 10¹⁴ CFU, and more particularly, preferably from 10⁸ to 10¹² CFU per day of lactic acid bacteria strains is administered. In specific embodiments, the repeated administration to a human or a animal of an effective amount of lactic acid bacteria strains is performed in combination with the administration of cannabinoid or opioid receptor agonists.

The compositions of the present invention may be administered by direct injection. The composition may be formulated for parenteral, mucosal, intramuscular, intravenous, subcutaneous, intraocular, intradermal or transdermal administration.

The term “administering” as used herein refers to administration of a composition of the present invention for the purposes of providing a medicament. In other words, in one embodiment the term “administering” means that the composition including bacteria is given (preferably as a medicament) to the subject, i.e. does not encompass the situation where the subject may comprise or acquire the bacteria present in the composition naturally.

The term “administered” includes delivery by delivery mechanisms including injection, lipid mediated transfection, liposomes, immunoliposomes, lipofectin, cationic facial amphiphiles (CFAs) and combinations thereof, or even viral delivery. The routes for such delivery mechanisms include but are not limited to mucosal, nasal, oral, parenteral, gastrointestinal, topical, or sublingual routes.

Preferably, in the present invention, administration is by an orally acceptable composition.

The mode of administration, the dose and the number of administrations can be optimised by those skilled in the art in a known manner.

### Process for Selecting a Microorganism

The invention also proposes a process for selecting a Lactobacillus strain(s) to prepare a support administered to humans or animals for an analgesic purpose in the gastrointestinal system comprising the following stages:
i) bringing the strain of Lactobacillus to be tested into contact with at least one epithelial cell;
ii) detecting the expression of the opioid receptors and/or cannabinoid receptors in least one epithelial cell;

In this process, suitably at least one epithelial cell of stage i) or ii) comes from the cell line ATCC HTB-38.

Stage ii) may be carried out by detecting the μ receptors for the opioids and/or the CB1 receptors and/or the CB2 receptors. Preferably, stage ii) is carried out by detecting the expression of the messenger RNA of the opioid receptors and/or cannabinoid receptors.

The invention can be applied to the treatment of pain affecting all organs, preferably organs having cannabinoid or opioid receptors and more preferably organs having mucosa.

These organs are for example spleen, digestive system including mouth and intestine, uterus, central nervous system, brain and skin.

One of the many embodiments of the invention which is related to the treatment of abdominal pain will be described hereafter. It must be understood that this embodiment is only illustrative of the invention and therefore the invention is not to be understood as being limited to this embodiment.

The gut microbiota is a complex ecosystem composed of hundreds of different bacterial species that together play an important role in the physiology of their host (Guarner F, Malagelada J R. Lancet 8, 512-519 (2003)). Use of gnotoxenic animals has provided compelling evidence regarding the benefit of the gut ecosystem on the metabolism of nutrients and organic substrates, the maturation of intestinal epithelium, vasculature, and lymphoid tissue, and the contribution to protective functions against pathogens (Falk P G et al. Microbiol. Mol. Biol. Rev. 62, 1157-1170 (1998)). The interest in probiotics and the modulation of microbiota for restoring and maintaining health continues to gain momentum. Experimental and clinical data suggest that changes in gut flora may be a basis for the variability of abdominal symptoms observed in functional gastrointestinal disorders and may be prevented by specific probiotic administration (Kajander K et al. Aliment. Pharmacol. Ther. 22, 387-394 (2005); O'Mahony L et al. Gastroenterology 128, 541-551 (2005)). We thus hypothesize that probiotics may induce expression of receptors on epithelial cells that locally control the transmission of nociceptive information to the intestinal nervous systems. Clear candidates were mu opioid (MOR) and cannabinoid 1 and 2 (CB1 and CB2) receptors, which are expressed in human small bowel and colon (Philippe D et al. Gut (Epub ahead of print); Massa F et al. J. Clin. Invest. 113, 1202-1209 (2004)), and have been shown in addition to their analgesic effect, to exert anti-inflammatory functions in several experimental models of colitis (Massa F et al. J. Clin. Invest. 113, 1202-1209 (2004); Stein C et al. Nat Med 8, 1003-1009 (2003); Philippe D et al. J. Clin. Invest. 111, 1329-1338 (2003); D'Argenio G et al. FASEB. J. 20, 568-570 (2006)).

The present invention is directed to methods of treating humans or animals by administering at least one microorganism and/or at least one metabolite thereof in an amount suitable for inducing an effect similar to cannabinoid or opioid receptor agonists.

Suitably administration of at least one strain of lactobacillus and/or a metabolite thereof may have an effect similar to 0.1 to 4 mg of morphine per kilogram of human or animal body, preferably 0.5 to 2 mg of morphine per kilogram of human or animal body, and more preferably around 1 mg of morphine per kilogram of human or animal body. Preferably, the at least one strain may be a Lactobacillus acidophilus or Lactobacillus salivarius strain or a combination thereof.

In specific embodiments, the Lactobacillus acidophilus strain is a strain registered at the ATCC under the number PTA-4797 or PTA-4800.

In certain aspects the methods of administering the at least one strain of lactobacillus and/or a metabolite thereof in combination with an opioid receptor agonist and/or cannabinoid receptor agonist are directed to inducing an analgesic or an anesthetic or an anti-inflammatory or an antidiarrheic effect or a regulatory effect on the metabolism or an antineoplasic effect mediated via the opioid receptors or cannabinoid receptors.

The compositions may be administered as a syrup, tablet, capsule; pomade, aerosol, ointment, enema, lotion, patch, confectionary, candy, lozenge or other classic pharmaceutical forms.

Combination with Other Components

A composition of the present invention may be used in combination with other components.

Examples of components which can be added to the composition include one or more of: thickeners, gelling agents, emulsifiers, binders, crystal modifiers, sweeteners (including artificial sweeteners), rheology modifiers, stabilisers, anti-oxidants, dyes, enzymes, carriers, vehicles, excipients, diluents, lubricating agents, flavouring agents, colouring matter, suspending agents, disintegrants, granulation binders, cholesterol reducing agents (such as sterols and stanols) etc. Preferably the other components include yeast extracts and magnesium ions (Mg²⁺). These other components may be prepared by use of chemical and/or enzymatic techniques and/or isolated from their natural environment.

As used herein the term “thickener or gelling agent” as used herein refers to an agent or a substance that prevents separation by slowing or preventing the movement of particles, either droplets of immiscible liquids, air or insoluble solids.

The term “stabiliser” as used here is defined as an ingredient or combination of ingredients that keeps a product (e.g. a water-in-oil product) from changing over time.

The term changing over time may be used in relation to for example colour changes, rehydration of the viable lactic acid micro-organism which may result in premature spoilage of product or general reduction of the shelf life of the water-in-oil product.

The term “emulsifier” as used herein refers to an ingredient or combination of ingredients (e.g. a water-in-oil product) that prevents the separation of emulsions.

“Carriers” or “vehicles” mean materials suitable for compound administration and include any such material known in the art such as, for example, any liquid, gel, solvent, liquid diluent, solubilizer, or the like, which is non-toxic and which does not interact with any components of the composition in a deleterious manner.

Examples of nutritionally acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

Examples of excipients include one or more of: microcrystalline cellulose and other celluloses, lactose, sodium citrate, calcium carbonate, dibasic calcium phosphate, glycine, starch, milk sugar and high molecular weight polyethylene glycols.

Examples of disintegrants include one or more of: starch (preferably corn, potato or tapioca starch), sodium starch glycollate, croscarmellose sodium and certain complex silicates.

Examples of granulation binders include one or more of: polyvinylpyrrolidone, hydroxypropylmethylcellulose (HPMC), hydroxypropylcellulose (HPC), sucrose, maltose, gelatin and acacia.

Examples of lubricating agents include one or more of: magnesium stearate, stearic acid, glyceryl behenate and talc.

The other components may be used simultaneously (e.g. when they are in admixture together or even when they are delivered by different routes) or sequentially (e.g. they may be delivered by different routes).

Preferably, when the composition of the present invention is admixed with any other components, the lactic acid micro-organism remains viable.

Preferably the lactic acid micro-organism as described herein becomes rehydrated and thus acquires the capacity to exert its nutritional and/or health benefits upon ingestion by the consumer.

As used herein the term “component suitable for animal or human consumption” means a component which is or can be added to the composition of the present invention as a supplement which may be of nutritional benefit, a fibre substitute or have a generally beneficial effect to the consumer. Preferably, the ingredients will be able to improve the shelf life of the product and stability of the viable culture.

The components may be prebiotics such as alginate, xanthan, pectin, locust bean gum (LBG), inulin, guar gum, galacto-oligosaccharide (GOS), fructo-oligosaccharide (FOS), lactosucrose, soybean oligosaccharides, palatinose, isomalto-oligosaccharides, gluco-oligosaccharides and xylo-oligosaccharides.

### Food

The composition of the present invention or the composition produced by a method according to the present invention may be used as—or in the preparation of—a food (i.e. a functional food). Here, the term “food” is used in a broad sense—and covers food for humans as well as food for animals (i.e. a feed). Preferably the food is for human consumption.

Suitably the food product may be a food supplement, a drink, a dairy product or a powder based on milk. Preferably the dairy product of animal or vegetable origin.

The term “dairy product” as used herein includes a medium comprising milk of animal and/or vegetable origin. As a milk of animal origin there can be mentioned cow's, sheep's buffalo's and goat's milk. As a milk of vegetable origin there can be mentioned any fermentable substance of vegetable origin which can be used according to the invention such as fermentable substances originating from soybeans, rice or cereals.

Suitably the dairy product may be a fermented milk or humanized milk.

As used herein, the term “functional food” means a product which is capable of providing not only a nutritional and or health effect but is also capable of delivering a further beneficial effect to the consumer.

Accordingly, functional foods are ordinary foods that have components or ingredients (such as those described herein) incorporated into them that impart to the food a specific functional—e.g. medical or physiological benefit—other than a purely nutritional effect.

Although there is no legal definition of a functional food most of the parties with an interest in this area agree that they are foods marketed as having specific health effects.

Some functional foods are nutraceuticals. Here, the term “nutraceutical” means a food which is capable of providing not only a nutritional effect and/or a taste satisfaction, but is also capable of delivering a therapeutic (or other beneficial) effect to the consumer. Nutraceuticals cross the traditional dividing lines between foods and medicine. Suitably a composition of the present invention may be—or may be used in the preparation of a nutraceutical.

When used as—or in the preparation of—a food—such as functional food—the composition of the present invention may be used in conjunction with one or more of: a nutritionally acceptable carrier, a nutritionally acceptable diluent, a nutritionally acceptable excipient, a nutritionally acceptable adjuvant, a nutritionally active ingredient.

### Food Ingredient

A composition of the present invention may be used as a food ingredient and/or feed ingredient.

As used herein the term “food ingredient” or “feed ingredient” includes a formulation which is or can be added to functional foods or foodstuffs as a nutritional supplement.

The food ingredient may be in the form of a solution or as a solid—depending on the use and/or the mode of application and/or the mode of administration.

### Pharmaceutical

The composition of the present invention may be used as—or in the preparation of—a pharmaceutical. Here, the term “pharmaceutical” is used in a broad sense—and covers pharmaceuticals for humans as well as pharmaceuticals for animals (i.e. veterinary applications). In a preferred aspect, the pharmaceutical is for human use and/or for animal husbandry.

The pharmaceutical can be for therapeutic purposes—which may be curative or palliative or preventative in nature. The pharmaceutical may even be for diagnostic purposes.

When used as—or in the preparation of—a pharmaceutical, the composition of the present invention may be used in conjunction with one or more of: a pharmaceutically acceptable carrier, a pharmaceutically acceptable diluent, a pharmaceutically acceptable excipient, a pharmaceutically acceptable adjuvant, a pharmaceutically active ingredient.

The pharmaceutical may be in the form of a solution or as a solid—depending on the use and/or the mode of application and/or the mode of administration.

Suitably the effect of a viable strain of Lactobacillus may be exerted following rehydration for example upon ingestion of the pharmaceutical comprising the composition as described herein.

In one aspect, the composition according to the present invention may be administered in an aerosol, for example by way of a nasal spray, for instance for administration to the respiratory tract.

In another aspect the composition according to the present invention may be advantageously be administered in an encapsulated form having a hard exterior or soft exterior and a liquid or fluid interior or in a chewable capsule. By way of example the hydrophobic combination may be incorporated in a fish oil capsule for ingestion.

### Pharmaceutical Ingredient

The composition may be used as pharmaceutical ingredients. Here, the composition may be the sole active component or it may be at least one of a number (i.e. 2 or more) active components.

The pharmaceutical ingredient may be in the form of a solution or as a solid - depending on the use and/or the mode of application and/or the mode of administration.

### Forms

The composition of the present invention may be used in any suitable form—whether when alone or when present in combination with other components or ingredients. Likewise, combinations comprising the composition of the present invention and other components and/or ingredients (i.e. ingredients—such as food ingredients, pharmaceutical ingredient or functional food ingredients) may be used in any suitable form.

The composition of the present invention may be used in the form of solid or liquid preparations or alternatives thereof. Examples of solid preparations include, but are not limited to tablets, capsules, dusts, granules and powders which may be wettable, spray-dried, freeze-dried or lyophilised. Examples of liquid preparations include, but are not limited to, aqueous, organic or aqueous-organic solutions, suspensions and emulsions. Preferably the liquid preparation as described herein is a water-in-oil preparation comprising a water-in-oil emulsion having a fat content of approximately from about 5% to about 98%.

Suitable examples of preparations include one or more of: tablets, pills, capsules, ovules, solutions or suspensions, which may contain flavouring or colouring agents, for immediate-, delayed-, modified-, sustained-, pulsed- or controlled-release applications.

By way of example, if the composition of the present invention is used in a tablet form—such for use as a functional ingredient—the tablets may also contain one or more of: excipients such as microcrystalline cellulose, lactose, sodium citrate, calcium carbonate, dibasic calcium phosphate and glycine; disintegrants such as starch (preferably corn, potato or tapioca starch), sodium starch glycollate, croscarmellose sodium and certain complex silicates; granulation binders such as polyvinylpyrrolidone, hydroxypropylmethylcellulose (HPMC), hydroxypropylcellulose (HPC), sucrose, gelatin and acacia; lubricating agents such as magnesium stearate, stearic acid, glyceryl behenate and talc may be included.

Examples of nutritionally acceptable carriers for use in preparations include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

Preferred excipients for the preparations include lactose, sucrose, maltose, starch, a cellulose, milk sugar or high molecular weight polyethylene glycols.

The forms may also include gelatin capsules; fiber capsules, fiber tablets etc.; or even fiber beverages.

The present invention will now be described further in the following numbered paragraphs:
1. Use of at least one microorganism and/or at least one metabolite thereof to prepare a support administered to humans or animals suitable for inducing an effect similar to cannabinoid or opioid receptor agonists.
2. Use according to paragraph 1, characterized in that said support has an effect similar to 0.1 to 4 mg of morphine per kilogram of human or animal body, preferably 0.5 to 2 mg of morphine per kilogram of human or animal body, and more preferably around 1 mg of morphine per kilogram of human or animal body.
3. Use according to one of the preceding paragraphs, characterized in that the microorganism is a Lactic acid bacteria strain and/or a probiotic strain.
4. Use according to paragraph 3, characterized in that the probiotic strain is a Lactobacillus acidophilus or Lactobacillus salivarius strain.
5. Use according to paragraph 4, characterized in that the Lactobacillus acidophilus strain is a strain registered at the ATCC under the number PTA-4797.
6. Use according to one of the preceding paragraphs, characterized in that an analgesic or an anaesthetic or an anti-inflammatory or an antidiarrheic effect or a regulatory effect on the metabolism or an antineoplasic effect is mediated via the opioid receptors or cannabinoid receptors.
7. Use according to one of the preceding paragraphs, characterized in that the support administered is a pharmaceutically acceptable support or a food product.
8. Use according to one of the preceding paragraphs, characterized in that the mode of administration of the support is oral, subcutaneous, mucosal, or cutaneous.
9. Use according to one of the preceding paragraphs, characterized in that the support administered is syrup, tablet, capsule; pomade, aerosol, ointment, enema, lotion, patch, confectionary, candy, lozenge or other classic pharmaceutical forms.
10. Use according to paragraphs 1 to 7, characterized in that the support administered is a dairy product of animal or vegetable origin.
11. Use of at least one microorganism and/or at least one metabolite thereof to prepare a support administered to humans or animals suitable for inducing an effect similar to cannabinoid or opioid receptor agonists in combination with cannabinoid or opioid receptor agonists.
12. Support administered to humans or animals comprising from 10⁶ to 10¹⁴ CFU, preferably 10⁸ to 10¹² CFU.
13. Method for inducing an effect similar to the effect of cannabinoid or opioid receptor agonists comprising repeatedly administering to a human or a animal an effective amount of microorganisms and/or at least one metabolite thereof preferably lactic acid bacteria strains and/or probiotic strains and more preferably Lactobacillus strains.
14. Method according to paragraph 12, characterized in that from 10⁶ to 10¹⁴ CFU, preferably from 10⁸ to 10¹² CFU per day of lactic acid bacteria strains is administered.
15. Method according to paragraph 12 or 13, characterized in that the repeatedly administration to a human or a animal of an effective amount of lactic acid bacteria strains is performed in combination with the administration of cannabinoid or opioid receptor agonists.
16. Process for selecting a microorganism to prepare a support administered to humans or animals for an analgesic purpose in the gastrointestinal system comprising the following stages:
i) bringing the microorganism to be tested into contact with at least one epithelial cell;
ii) detecting the expression of the opioid receptors and/or cannabinoid receptors in least one epithelial cell;
17. Process according to paragraph 15 characterized in that at least one epithelial cell of stage i) or ii) comes from the cell line ATCC HTB-38.
18. Process according to one of paragraphs 15 to 16 characterized in that stage ii) is carried out by detecting the μ receptors for the opioids and/or the CB1 receptors and/or the CB2 receptors.
19. Process according to one of paragraphs 15 to 16 characterized in that stage ii) is carried out by detecting the expression of the messenger RNA of the opioid receptors and/or cannabinoid receptors

### DESCRIPTION OF THE DRAWINGS

FIG. 1: Visceral perception evaluated by pain threshold (mean±SEM). Evaluation A) in rats (n=20) receiving or not (saline) enema of butyrate and treated or not (Ctl) with the NCFM strain (10⁹ CFU/day during 15 days), and B) in rats (n=20) with colonic hypersensitivity induced by butyrate enemas and treated or not with escalating subcutaneous dosages of morphine;

FIG. 2 illustrates the expression of MOR, CB1 and CB2 by rat colonic epithelium cells in response to exposure to L. acidophilus NCFM. Left column: CMC=carboxymethylcellulose (control); Right column: NCFM (L. acidophilus NCFM); Upper line: detection of MOR; Middle line: detection of CB1; Lower line: detection of CB2;

FIG. 3 illustrates pain threshold in rats with colonic hypersensitivity receiving NCFM 10⁹ CFU/d during 15 days, morphine (0.1 mg/kg), subcutaneously, 30 min before colorectal distension or NCFM and morphine together; and

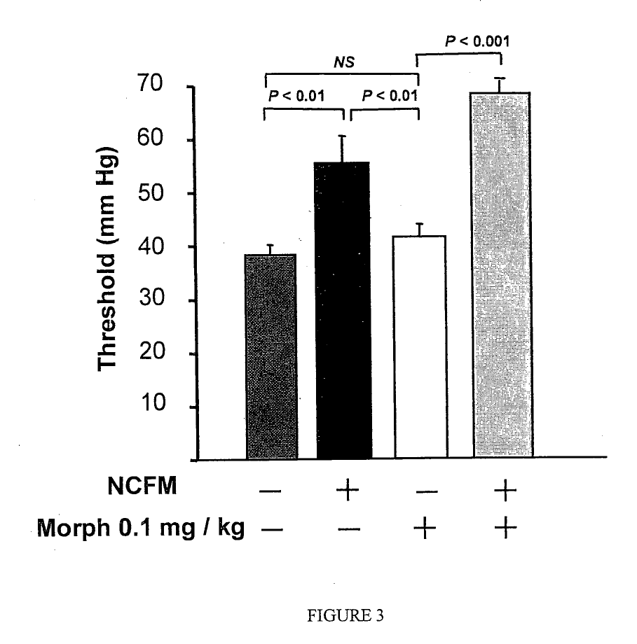

FIG. 4 illustrates pain threshold in control rats with colonic hypersensitivity receiving NCFM (109 CFU/d, 15 days) or control (Ctl) and treated either with CB2 antagonist (AM630), a MOR antagonist (naloxone methiodide NLX) or neither. AM630 and NLX were used at optimal dosage and injected peritoneally 30 min before colorectal distension.

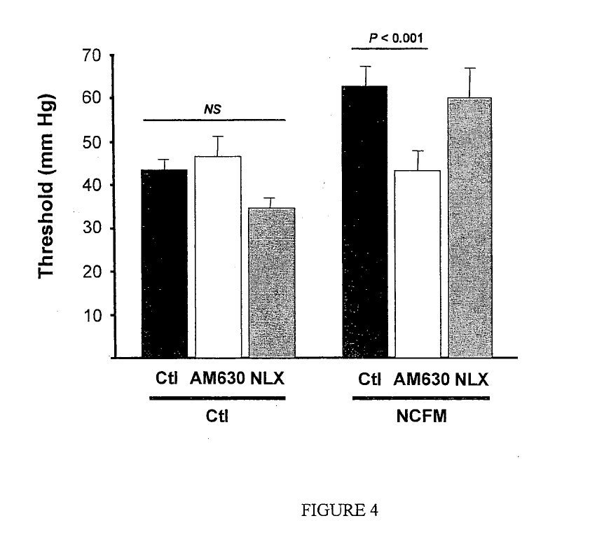

### EXAMPLES

The invention will now be further described by way of Examples, which are meant to serve to assist one of ordinary skill in the art in carrying out the invention and are not intended in any way to limit the scope of the invention.

### Example 1

To determine the functional role of NCFM-induced analgesic receptors, we assessed the visceral perception of rats using an established technique of colorectal distension after inflation of a balloon inserted intrarectally and connected to a barostat system (Kajander K et al. Aliment. Pharmacol. Ther. 22, 387-394 (2005); Bourdu S et al. Gastroenterology 128, 1996-2008 (2005)).

Example: The protocol used is the following:

### Animals

Male Sprague-Dawley rats (Charles River, l'Arbresle, France) weighing 175-200 g were used in this study. Rats were maintained in laboratory conditions for 1 week before experiment. The animals were housed 5 per cage with food and water available ad libitum. All studies were performed in accordance with the proposal of the committee for Research and Ethical Issues of the International Association for the Study of Pain (Zimmermann M, Pain 1983; 16:109-110). Great care was taken, particularly with regard to housing conditions, to avoid or minimize discomfort to the animals.

### Induction of Colonic Hypersensitivity by Butyrate Enemas:

For each enema, a catheter (2-mm Fogarty catheter) was placed in the colon at 7 cm from the anus, and the animals received 1 ml of 200 mM of sodium butyrate at neutral pH (pH6.9) twice daily for 3 days. Healthy animals received saline.

### Evaluation of Colonic Sensitivity

Nociception in the animals was assessed by measuring the intracolonic pressure required to induce a behavioral response during colorectal distension (CRD) due to the inflation of a balloon introduced in the colon. This response was characterized by an elevation of the hind part of the animal body and clearly visible abdominal contraction corresponding to the severe contractions (Al Chaer, gastro 2000; Tarrerias, pain 2002; Bourdu et al., 2005). Briefly, rats were anesthetized with volatile anaesthesia (2% isoflurane), the balloon (prepared as previously described in Bourdu & al, 2005) was inserted intrarectally in a minimally invasive manner to 7 cm from the anus, and the catheter was taped to the base of the tail. After 5 minutes, rats were placed in the middle of a 40×40-cm Plexiglas box and the catheter was connected to an electronic barostat apparatus (Synectics Visceral Stimulator; Medtronic, Boulogne-Billancourt, France). Increasing pressure was continuously applied until pain behaviour was displayed or a cutoff pressure of 80 mm Hg was reached.

### Treatments of Animals by Probiotics

Four groups of animals were used (n=10/group). One group of healthy animals and one treated with butyrate received once a day during fifteen days by gastric gavage, 10⁹ CFU of NCFM strain and the two other groups of healthy and butyrate-treated animals received CarboxyMethyl Cellulose (CMC) by the same route and during the same time. NCFM strain was resuspended in 0.5% CMC (CarboxyMethyl Cellulose, Sigma). Butyrate or saline instillations began 7 days after the first gavage for three days. Colonic hypersensitivity was determined 14 days after the after beginning of oral treatment thus 7 days after colonic instillations.

### Treatments of Animals by Morphine

Seven days after the beginning of butyrate instillations, 3 experimental series corresponding to the different treatments were performed. Animals were treated with morphine (0.03, 0.1, 0.3, 1, 3, and 10 mg/kg subcutaneously). CRD tests were performed 30 minutes after the injections

### Experimental Protocols, Expression of Results and Statistical Analysis

All experiments were performed in a blind manner by the same experimenter using the block method. Results are expressed as mean±SEM of raw data. Results of CRD testing were analysed using one-way analysis of variance (ANOVA) followed of a Bonferroni post-hoc test to compare several treatments. Differences were considered significant at P<0.05.

### Results

### Effect of NCFM on Colonic Sensitivity in Healthy Rats and in Butyrate-Induced Colonic Hypersensitivity

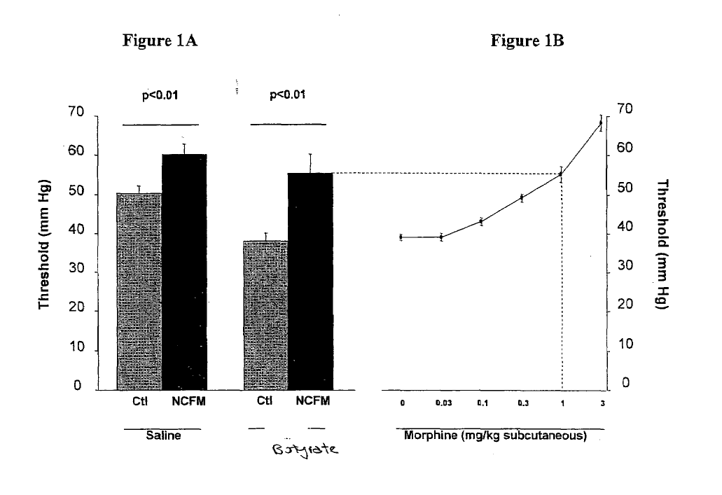

In healthy rats receiving oral CMC, the colonic reaction threshold that induces clearly visible abdominal contraction during colorectal distension (CRD) experiment is of 50.4±1.9 mmHg and corresponds to normal threshold of reaction (50.6±2.1 in Bourdu et al., 2005). Administration of NCFM in healthy rats induced a significant increase of this threshold to 60.2±2.8 mmHg (p<0.01 vs rats treated with CMC) (FIG. 1A).

Butyrate enemas induced a significant decrease in CRD threshold (38.3±1.9, p<0.001 vs healthy animals receiving oral CMC) which corresponded to the colonic hypersensitivity observed by Bourdu et al. (2005) with the same butyrate treatment. In butyrate-treated animals, NCFM increases the CRD threshold to a normal threshold of 55.5±5.1 mmHg (p<0.01 versus butyrate-treated animals receiving oral CMC) (FIG. 1A).

Morphine is known to induce a dose-dependent decrease of the colonic hypersensitivity in rat with visceral hypersensitivity induced by the butyrate assessed by the CRD test (Bourdu et al). The analgesic effect of NCFM was compared to the effect induces by morphine injection in the DCR model. Administration of NCFM has an equivalent effect of an injection of morphine at the dosage of 1 mg/kg (FIG. 1B).

To summarize, compared to untreated rats where a mean colorectal distension of 50±2 mmHg was required to induce pain characterized by clear visible abdominal contraction and elevation of the hind part of the animal body (Bourdu S et al. Gastroenterology 128, 1996-2008 (2005)), oral administration of the NCFM strain (10⁹ CFU/day) during 15 days decreased the normal visceral perception allowing a 20% increase of this pain threshold to 60±2.8 mmHg (p<0.01)(FIG. 1A). Similarly, in a model of chronic colonic hypersensitivity elicited by butyrate enemas and mimicking irritable bowel syndrome (Bourdu S et al. Gastroenterology 128, 1996-2008 (2005)), colonic hypersensitivity of rats was improved by the NCFM strain which increased by 44% the colorectal distension threshold to 55.5±5 mmHg vs 38.3±1.9 in untreated animals (p<0.01)(FIG. 1A). In this model, pharmacologic investigations using escalating dosages of a major pain regulator revealed that NCFM mediated a similar antinociceptive effect than 1 mg/kg of morphine administered subcutaneously (FIG. 1B).

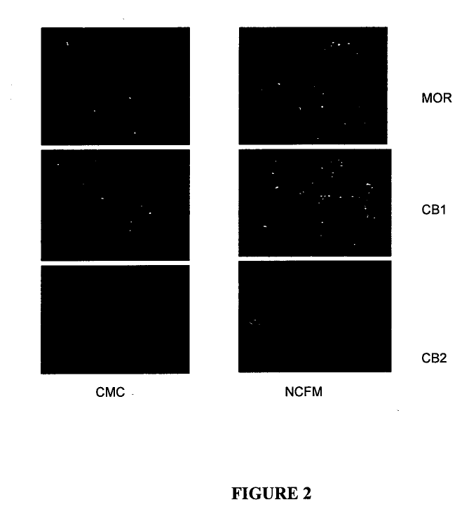

In the same study, immunohistology of the rat colonic epithelium cells was performed to detect expression of MOR, CB1 and CB2 in response to exposure to L. acidophilus NCFM (FIG. 2).

### MOR-CB1-CB2 Immunohistochemistry: Protocol

Immunohistochemistry was performed on colon embedded-paraffin sections of rat receiving the NCFM strain. Untreated animals were used as controls. After permeabilisation during 5 min in PBS containing 0.1% triton X-100 at 4° C., sections were incubated for 15 min with 1.5% goat normal serum and 15 min with blocking buffer (1% BSA in milk) to minimize non-specific adsorption of the antibodies. The tissues were subsequently incubated with the rabbit polyclonal primary antibody directed against CB1 (1:200, Cayman Chemical, Ann Arbor, USA) or CB2 (1:10, Alpha Diagnostic, San Antonio, USA) or MOR (1:500, Diasorin, Antony, France) for 2 to 12 hours at room temperature. Sections were then incubated for 1 h at room temperature with Alexa 488 goat anti-rabbit IgG conjugated to FITC fluorochrome (dilution 1:100, Dako Laboratories, Trappes, France). Between each stage, sections were rinsed twice for 5 min in PBS containing 0.05% triton X-100. Then slides were counterstained with Hoescht solution (0.125 mg/mL) and mounted for microscopy. Negative controls consisted of staining with normal rabbit serum instead of specific antibody. Immunofluorescence was revealed under a fluorescence microscope (Leica, Bensheim, Germany).

### Results

Evaluation of NCFM administration on the expression of mu opioid and cannabinoid receptors at the protein level in the rat colon was evaluated by immunohistochemistry using specific antibodies directed respectively against MOR, CB1 and CB2.

The expression of CB1, CB2 and MOR was increased in the colonic mucosa of rats receiving NCFM (10⁹ bacteria during 15 days) compared to control rats treated only with the vehicle (CMC, 0.5% Carboxymethyl cellulose). Indeed, the analgesic effect of NCFM is associated to the increased expression of MOR, CB1 and CB2.

Visceral pain is a prominent symptom of many clinical disorders, traditionally viewed as transmitted by single neural mechanisms (Cervero F, Laird J M A. Lancet 353, 2145-2148 (1999)). The present results advance our understanding of visceral pain by showing that direct contact of specific Lactobacillus NCFM strains on epithelial cells is able to induce through the NFκB pathway MOR, CB1 and CB2 expression and to contribute to the modulation and restoration of normal visceral pain perception. Reasons of the particular functions of this strain remain unknown. L. acidophilus NCFM is a well known probiotic, isolated from human faeces, and widely investigated for its physiological, biochemical and fermentative properties (Sanders M E, Klaenhammer T R. J. Dairy. Sci. 84; 319-331 (2001)). Since complete genome sequence of L. acidophilus NCFM revealed unique features compared to other probiotic genomes (Altermann E et al. Proc. Natl. Acad. Sci. 102, 3906-3912 (2005)), further investigations will determine their potential involvement in the regulation of MOR and CBs expression.

NCFM for example, given orally compared to classical dosage of morphine (1 mg/kg) administered subcutaneously suggests that specific modulation of intestinal flora may be a promising safe and relatively inexpensive new treatment of pain, particularly in patients with irritable bowel syndrome, a disease affecting 20% of the general population and characterized by an abdominal hypersensitivity.

Hence, microorganisms such as probiotics, for example NCFM, can be used as a treatment for pain as a substitute of morphine or other drugs having an effect on cannabinoid or opioid receptors (cannabinoid or opioid agonists). Preliminary experiments enable to conclude that such effect could be obtained as well with other Lactobacillus acidophilus strains, Lactobacillus salivarius and Lactobacillus casei strains.

The application of lactic acid bacteria according to the invention on other mucosa, for example cutaneous application may be a promising and safe new treatment of pain.

### Example 2

### Effect of Probiotic Co-Administrated with a Suboptimal Dosage of Morphine in Colorectal Distension Test in Rats with Colonic Hypersensitivity Induced by Butyrate

### Animals

Male Sprague-Dawley rats (Charles River, l'Arbresle, France) weighing 175-200 g were used in this study. Rats were maintained in laboratory conditions for 1 week before experiment. The animals were housed 5 per cage with food and water available ad libitum. All studies were performed in accordance with the proposal of the committee for Research and Ethical Issues of the International Association for the Study of Pain (Zimmermann M, Pain 1983; 16:109-110). Great care was taken, particularly with regard to housing conditions, to avoid or minimize discomfort to the animals.

### Induction of Colonic Hypersensitivity by Butyrate Enemas:

For each enema, a catheter (2-mm Fogarty catheter) was placed in the colon at 7 cm from the anus, and the animals received 1 ml of 200 mM of sodium butyrate at neutral pH (pH6.9) twice daily for 3 days. Healthy animals received saline.

### Evaluation of Colonic Sensitivity

Nociception in the animals was assessed by measuring the intracolonic pressure required to induce a behavioral response during colorectal distension (CRD) due to the inflation of a balloon introduced in the colon. This response was characterized by an elevation of the hind part of the animal body and clearly visible abdominal contraction corresponding to the severe contractions (Al Chaer, gastro 2000; Tarrerias, pain 2002; Bourdu et al., 2005). Briefly, rats were anesthetized with volatile anaesthesia (2% isoflurane), the balloon (prepared as previously described in Bourdu & al, 2005) was inserted intrarectally in a minimally invasive manner to 7 cm from the anus, and the catheter was taped to the base of the tail. After 5 minutes, rats were placed in the middle of a 40×40-cm Plexiglas box and the catheter was connected to an electronic barostat apparatus (Synectics Visceral Stimulator; Medtronic, Boulogne-Billancourt, France). Increasing pressure was continuously applied until pain behaviour was displayed or a cutoff pressure of 80 mm Hg was reached.

### Treatment of Animals by Probiotics

Four groups of animals treated with butyrate were used (n=10/group). Two groups were treated once a day during fifteen days by gastric gavage, 10⁹ CFU of NCFM strain and the two other groups of butyrate-treated animals received CarboxyMethyl Cellulose (CMC) by the same route and during the same time. NCFM strain was resuspended in 0.5% CMC (CarboxyMethyl Cellulose, Sigma). Butyrate instillations began 7 days after the first gavage for three days. Colonic hypersensitivity was determined 14 days after the after beginning of oral treatment thus 7 days after colonic instillations.

### Treatments of Animals by Morphine

Seven days after the beginning. of butyrate instillations, animals were treated with a sub-efficient dosage of morphine (0.1 mg/kg subcutaneously) or with a saline injection. CRD tests were performed 30 minutes after the injections.

### Experimental Protocols Expression of Results and Statistical Analysis

All experiments were performed in a blind manner by the same experimenter using the block method. Results are expressed as mean±SEM of raw data. Results of CRD testing were analysed using one-way analysis of variance (ANOVA) followed of a Bonferroni post-hoc test to compare several treatments. Differences were considered significant at P<0.05.

### Results

### Lactobacillus Acidophilus NCFM Potentiates the Effect of Suboptimal Dosage of Morphine on Visceral Pain

No difference on pain threshold between rats with colonic hypersensitivity induced butyrate receiving the suboptimal dosage of morphine (0.1 mg/kg) or not was obtained. NCFM enhanced by 65% the suboptimal analgesic effects of morphine used at 0.1 mg/kg (68.3±2.64 vs 41.5±2.4, p<0.001).

### Example 3

### Evaluation of the Functional Role of CB2 and MOR Receptor in the Analgesia Induced by NCFM

### Animals

Male Sprague-Dawley rats (Charles River, l'Arbresle, France) weighing 175-200 g were used in this study. Rats were maintained in laboratory conditions for 1 week before experiment. The animals were housed 5 per cage with food and water available ad libitum. All studies were performed in accordance with the proposal of the committee for Research and Ethical Issues of the International Association for the Study of Pain (Zimmermann M, Pain. 1983; 16:109-110). Great care was taken, particularly with regard to housing conditions, to avoid or minimize discomfort to the animals.

### Induction of Colonic Hypersensitivity by Butyrate Enemas:

For each enema, a catheter (2-mm Fogarty catheter) was placed in the colon at 7 cm from the anus, and the animals received 1 ml of 200 mM of sodium butyrate at neutral pH (pH6.9) twice daily for 3 days. Healthy animals received saline.

### Evaluation of Colonic Sensitivity

Nociception in the animals was assessed by measuring the intracolonic pressure required to induce a behavioral response during colorectal distension (CRD) due to the inflation of a balloon introduced in the colon. This response was characterized by an elevation of the hind part of the animal body and clearly visible abdominal contraction corresponding to the severe contractions (Al Chaer, gastro 2000; Tarrerias, pain 2002; Bourdu et al., 2005). Briefly, rats were anesthetized with volatile anaesthesia (2% isoflurane), the balloon (prepared as previously described in Bourdu & al, 2005) was inserted intrarectally in a minimally invasive manner to 7 cm from the anus, and the catheter was taped to the base of the tail. After 5 minutes, rats were placed in the middle of a 40×40-cm Plexiglas box and the catheter was connected to an electronic barostat apparatus (Synectics Visceral Stimulator; Medtronic, Boulogne-Billancourt, France). Increasing pressure was continuously applied until pain behaviour was displayed or a cutoff pressure of 80 mm Hg was reached.

### Treatments of Animals by Probiotics

Four groups of animals treated with butyrate were used (n=10/group). Two groups were treated once a day during fifteen days by gastric gavage, 10⁹ CFU of NCFM strain and the two other groups of butyrate-treated animals received CarboxyMethyl Cellulose (CMC) by the same route and during the same time. NCFM strain was resuspended in 0.5% CMC (CarboxyMethyl Cellulose, Sigma). Butyrate instillations began 7 days after the first gavage for three days. Colonic hypersensitivity was determined 14 days after the after beginning of oral treatment thus 7 days after colonic instillations.

### Treatments of Animals by Specific MOR and CB2 Antagonists

Seven days after the beginning of butyrate instillations, animals were treated with the specific CB2 antagonist (AM630, 3 g/kg, Tocris), or with peripheric MOR antagonist (Naloxone methiodide, 2 mg/kg, Sigma). A saline injection was performed in control rats. CRD tests were performed 30 minutes after the injections.

### Experimental Protocols Expression of Results and Statistical Analysis

All experiments were performed in a blind manner by the same experimenter using the block method. Results are expressed as mean±SEM of raw data. Results of CRD testing were analysed using one-way analysis of variance (ANOVA) followed of a Bonferroni post-hoc test to compare several treatments. Differences were considered significant at P<0.05.

### Results

Preponderant Role of CB2 in the Control of Abdominal Pain Induced by Lactobacillus acidophilus NCFM.

In control rat, having a visceral hypersensitivity induced by the butyrate, the CB2 (AM630) or MOR (NLX) receptor antagonists have no effect on pain perception.

NCFM-induced analgesia was significantly inhibited by peritoneal administration of the cB2-selective antagonist (AM-630) but not by the opioid receptor antagonist naloxone methiodide, providing indirect evidence for a physiological role of CB2 in the control of intestinal pain.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

### REFERENCES

1. Guarner F, Malagelada J R. Lancet 8, 512-519 (2003).
2. Falk P G et al. Microbiol. Mol. Biol. Rev. 62, 1157-1170 (1998).
3. Kajander K et al. Aliment. Pharmacol. Ther. 22, 387-394 (2005).
4. O'Mahony L et al. Gastroenterology 128, 541-551 (2005).
5. Philippe D et al. Gut (Epub ahead of print).
6. Massa F et al. J. Clin. Invest. 113, 1202-1209 (2004).
7. Stein C et al. Nat Med 8, 1003-1009 (2003).
8. Philippe D et al. J. Clin. Invest. 111, 1329-1338 (2003).
9. D'Argenio G et al. FASEB. J. 20, 568-570 (2006).
10. Bourdu S et al. Gastroenterology 128, 1996-2008 (2005).
11. Cervero F, Laird J M A. Lancet 353, 2145-2148 (1999).
12. Sanders M E, Klaenhammer T R. J. Dairy. Sci. 84; 319-331 (2001).
13. Altermann E et al. Proc. Natl. Acad. Sci. 102, 3906-3912 (2005).

1. A composition comprising:
i) at least one strain of a Lactobacillus sp and/or a metabolite thereof; and
ii) a cannabinoid receptor agonist and/or a opioid receptor antagonist.

2. A composition according to claim 1 wherein the composition is a food composition and optionally further comprises a food ingredient.

3. A composition according to claim 1 wherein the composition is a pharmaceutical composition and optionally further comprises a pharmaceutically acceptable carrier, diluent or excipient.

4. A composition according to claim 1 wherein the at least one strain of a Lactobacillus sp and/or a metabolite thereof is a Lactobacillus acidophilus strain and/or a metabolite thereof, a Lactobacillus salivarius strain and/or a metabolite thereof, and/or a Lactobacillus casei strain and/or a metabolite thereof.

5. A composition according to claim 1 wherein the at least one strain of a Lactobacillus sp and/or a metabolite thereof is a Lactobacillus acidophilus strain and/or a metabolite thereof.

6. A composition according to claim 1 wherein said Lactobacillus acidophilus strain and/or a metabolite thereof is Lactobacillus acidophilus NCFM and/or a metabolite thereof.

7. A composition according to claim 1 wherein the at least one strain of a Lactobacillus salivarius and/or a metabolite thereof is Lactobacillus salivarius LS 33 and/or a metabolite thereof.

8. A composition according to claim 1 wherein the at least one strain of a Lactobacillus sp is present in the amount of 10⁶ to 10¹⁴ CFU, preferably 10⁸ to 10¹² CFU.

9. A method of treating and/or ameliorating any one or more of the group selected from: pain, inflammation, neoplasism and diarrhea comprising administering an effective amount of a composition according to claim 1.

10. A composition according to claim 1 for use as a medicament.

11. Use of a composition according to claim 1 as one or more of the following selected from the group consisting of: an analgesic, an anti-inflammatory, an anti-diarrheic and an anti-neoplastic.

12. Use according to claim 11 wherein said use is as an analgesic.

13. Use of a composition according to claim 1 in the manufacture of a medicament for treating and/or ameliorating the perception of pain.

14. Use according to claim 13 when said pain is pain associated with irritable bowel syndrome and/or irritable bowel disease.

15. Use of a composition according to claim 1 in the manufacture of a medicament for treating and/or ameliorating diarrhea.

16. Use of a composition according to claim 1 in the manufacture of a medicament for treating and/or ameliorating inflammation.

17-19. (canceled)
